(19) United States
(12) Patent Application Publication
(10) Pub. No.: US 2024/0124373 A1
(43) Pub. Date: Apr. 18, 2024

(54) CO CONVERSION CONTROL FOR MULTISTAGE FISCHER-TROPSCH SYNTHESES

(71) Applicant: INERATEC GMBH, Karlsruhe (DE)

(72) Inventor: Julian BAUDNER, Karlsruhe (DE)

(21) Appl. No.: 18/555,047

(22) PCT Filed: Apr. 13, 2022
(86) PCT No.: PCT/EP2022/059829

§ 371 Date: Oct. 12, 2023

(30) Foreign Application Priority Data
Apr. 27, 2021 (DE) 10 2021 110 735.0

(51) Int. Cl. C07C 1/04; B01J 8/04; C07C 2/04

(52) U.S. Cl. CPC C07C 1/0435; B01J 8/0492; C07C 1/046; C07C 2/04

(57) ABSTRACT

The present invention relates to methods for operating Fischer-Tropsch syntheses for the production of long-chain hydrocarbons and to plants for carrying out these processes, wherein the CO conversion is controlled and/or the catalyst deactivation is compensated.

All documents cited in the present application are incorporated by reference in their entirety into the present disclosure.

The present invention relates to methods for operating Fischer-Tropsch syntheses for the production of long chain hydrocarbons and to plants for carrying out these methods, whereby the CO conversion is controlled and/or the catalyst deactivation is compensated.

The process of the Fischer-Tropsch synthesis (FTS) used to produce hydrocarbons has been known for many decades. In this process, a synthesis gas consisting predominantly of carbon monoxide (CO) and hydrogen (H₂) is converted to hydrocarbons by heterogeneous catalysis in a synthesis reactor. The products in the outlet stream of such a synthesis reactor essentially comprise four fractions:
1.) A gas phase consisting of unreacted synthesis gas (CO, H₂), short-chain hydrocarbons and volatile components of the by-products, as well as optionally inert gases, such as N₂ and CO₂.
2) A waxy phase solid at ambient temperature and pressure of hydrocarbons.
3) A hydrophobic phase liquid at ambient temperature and pressure of hydrocarbons.
4) An aqueous phase of reaction water forming and organic compounds dissolved therein.

The synthesis gas for such FTS comes, for example, from gasification of biomass, from synthesis gas generation from fossil starting materials (natural gas, crude oil, coal), or from electricity-based processes (conversion of electrolytically generated H₂ as well as CO₂).

A central characteristic of FTS is the fact that a very broad product spectrum (from C₁ to >C₁₀₀) is always produced. Depending on the application, the increase in selectivity of a certain main product (from fuels to chemical value products) is of interest. Long-chain, waxy hydrocarbons can, inter alia, be fed to industry for material use, or serve in the conventional refinery process as a feedstock for high-quality fuels with a low CO₂ footprint. However, the proportion of this wax phase, one of the highest-value products of synthesis, is only in the range of a few percent.

Known processes for the production of long-chain hydrocarbons using FTS are fraught with disadvantages.

One major problem is the triangular dilemma of conversion, selectivity and catalyst deactivation, whereby two dependencies of FT product formation can only be achieved or maximized at the expense of the third.

In addition, it is problematic that if the reactants are fed to the reactor in a non-stoichiometric ratio (H₂/CO) in relation to the reaction and are largely converted in the reactor, the effect of non-stoichiometry is amplified in the outlet of the reactor. For example, an over-stoichiometric ratio at the inlet of the reactor leads to an even more over-stoichiometric ratio at the outlet. The same applies to a sub-stoichiometric ratio of H₂/CO; the ratio of H₂/CO continues to rise or fall over the progress of the reaction in the reactor up to a situation where the component that was less present is completely consumed.

This in turn leads to the following problems: over-stoichiometric ratios of hydrogen to carbon monoxide lead to the increased formation of unwanted short-chain and thus gaseous products, resulting in a reduced yield of target products. Sub-stoichiometric ratios of hydrogen to carbon monoxide lead to increased formation of target products, but lack of hydrogen, with almost complete hydrogen consumption, leads to greater and faster deactivation of the catalyst due to coke formation on the catalyst as well as possibly its re-oxidation.

Particularly problematic in the prior art is that the CO conversion rate cannot be controlled and maintained precisely enough without too much hydrogen being consumed and carbon deposits arising or possibly the catalyst being re-oxidised. Furthermore, it is a problem of the prior art that the catalysts lose activity over time and thus reduce the conversion.

Also known, for example, from U.S. Pat. No. 7,795,318 B2 are plants and processes for multi-stage Fischer-Tropsch synthesis, in which a synthesis gas mixture is added to each individual synthesis reactor via a respective individual mixing apparatus. From WO 2004/050799 A1 it is known to control Fischer-Tropsch syntheses via the space velocity, i.e. the weight volume flow, of the gases passed through, i.e. to continuously adjust the space velocity/the weight volume flow. In addition, certain gas-permeable catalyst structures are required there.

Accordingly, the object of the present invention was to overcome the above-described disadvantages of the prior art and to provide a method for operating an FTS with which the above problems can be effectively countered.

Further objects arise for the skilled person when considering the claims and from the following description.

These and further objects arising for the person skilled in the art from the present description are solved by the subject matter shown in the claims, whereby the dependent claims represent preferred and particularly advantageous embodiments.

In the context of the present invention, all indications of quantity are to be understood as indications of weight, unless otherwise indicated.

In the context of the present invention, the term “ambient temperature” means a temperature of 20° C. Temperature indications are in degrees Celsius (° C.) unless otherwise indicated.

Unless otherwise stated, the reactions or process steps indicated are carried out at ambient pressure (=normal pressure/atmospheric pressure), i.e. at 1013 mbar.

Pressure data in the context of the present invention, unless otherwise stated, mean absolute pressure data, i.e. x bar means x bar absolute (barₐ) and not x bar gauge.

Under long-chain hydrocarbons are understood herein hydrocarbons with at least 25 carbon atoms (C₂₅). The long-chain hydrocarbons with at least 25 carbon atoms can be linear or branched.

Under shorter-chain hydrocarbons are understood herein hydrocarbons with 5 to 24 carbon atoms (C₅-C₂₄). The shorter-chain hydrocarbons with 5 to 24 carbon atoms can be linear or branched.

Under short-chain hydrocarbons are understood herein hydrocarbons with 1 to 4 carbon atoms (C₁-C₄). The short-chain hydrocarbons with 4 carbon atoms can be linear or branched.

In the context of the present invention, the term “comprising” can in particular also mean “consisting of”. In this respect, a formulation “comprising element “A” and element “B”” is to be interpreted in such a way that further elements (“C”, “D”, . . . ) are permitted, but also that in a preferred embodiment only the elements “A” and “B” may be present.

In particular, a subject matter of the present invention is a method of operating a Fischer-Tropsch synthesis comprising the steps of,
I) feeding a synthesis gas containing H₂ and CO into a first fixed-bed synthesis reactor comprising a first catalyst bed to form hydrocarbons by catalytic reaction,
II) feeding a product stream exiting the first fixed-bed synthesis reactor comprising hydrocarbons to a product separation to separate a fraction of hydrocarbons from the product stream,
III) feeding the remaining fraction of the product stream comprising short chain and shorter chain hydrocarbons to a second fixed-bed synthesis reactor comprising a second catalyst bed to form long chain hydrocarbons by catalytic reaction,
wherein synthesis gas is added exclusively to the first fixed-bed synthesis reactor, wherein further
   the weight volume flow of synthesis gas introduced into the first fixed-bed synthesis reactor is adjusted to a value and kept constant at this value during the process,
   the molar H₂:CO ratio in the synthesis gas is adjusted from 1.7:1 to 2.3:1,
   the inert gas content in the synthesis gas is between 0 and 40 vol. %,
   the same cobalt-based Fischer-Tropsch catalyst is used in both reactors,
   the weight ratio of the amount of catalyst in the first fixed-bed synthesis reactor to the amount of catalyst in the second fixed-bed synthesis reactor is set to between 1.1:1 and 4.3:1,
   the first fixed-bed synthesis reactor is operated at a pressure of 10 to 50 bar, and the second fixed-bed synthesis reactor is operated at a pressure of 10 to 50 bar,
characterized in that
the reactor temperature is controlled to an equal value between 180° C. and 250° C. depending on the desired total CO conversion in both synthesis reactors, which is between 40 and 90 mol %, and in that the hydrogen conversion, considered over all stages, is at most 99 mol %.

The target products that are produced by the method according to the invention preferably comprise the solid, waxy phase as well as the liquid, hydrophobic phase, but in particular the solid, waxy phase of hydrocarbons. Inter alia, these can be supplied to industry for material use, or used in the conventional refinery process as a starting product for high-quality fuels.

The method according to the invention has, among others the advantage that the yield of long-chain hydrocarbons is increased. For this purpose, the synthesis gas is first fed into the first fixed-bed synthesis reactor. A part of the synthesis gas reacts under Fischer-Tropsch conditions to form hydrocarbon compounds. In a downstream product separation, parts of the hydrocarbons are separated from the rest of the material stream.

The products remaining in the stream, which leave the product separation, are fed to the second fixed-bed synthesis reactor. The material stream fed to the second fixed-bed synthesis reactor thus preferably consists of hydrocarbons, preferably short-chain and/or shorter-chain hydrocarbons, residual reaction water, unreacted synthesis gas and by-products of the first synthesis, as well as impurities (e.g. N₂).

In the second fixed-bed synthesis reactor, in addition to the synthesis of new hydrocarbons, the growth of the previously synthesized short and/or shorter-chain hydrocarbons into long-chain alkanes and/or alkenes is thus also promoted.

Overall, the yield of long-chain hydrocarbons can thus be increased with the method according to the invention.

The synthesis reactors used in the method according to the invention are fixed-bed synthesis reactors. A fixed-bed synthesis reactor in the sense of the present invention is a reactor in which at least one, preferably exactly one, bed of catalyst particles is arranged. For this purpose, a support (mounting), on which the catalyst is arranged, may be provided in its interior. The reactor is flowed through by gases and/or liquids (fluids) to be reacted, the reaction takes place at the catalyst (contact) (heterogeneous catalysis).

The architecture of the first and second fixed-bed synthesis reactor is not limited in principle. Preferably, the first and second fixed-bed synthesis reactors have essentially the same architecture.

In preferred embodiments of the present invention, the fixed-bed synthesis reactors used are preferably microstructured fixed-bed synthesis reactors. This allows the size of the overall plant to be varied to a much greater extent than in the plant concepts presented so far.

Microstructured reactors are preferably characterised by the fact that they have a large inner surface and can thus ensure particularly efficient heat transfer. By that exothermic or endothermic reactions in particular can be operated in a well-controlled manner. In a generally accepted but not legally binding definition, the internal structures of microstructured reactors are smaller than 1 mm in at least one dimension.

Particularly well-suited in the context of the present invention are microreactors such as those described, for example, in DE 10 2015 111 614 A1, in particular paragraphs [0023] to [0028] and FIGS. 1 to 4.

In particular, the present invention does not use reactors with catalyst structures as described in WO 2004/050799 A1, since these are large catalyst structures that do not comprise individual catalyst particles.

In preferred embodiments of the present invention, a fixed-bed synthesis reactor may comprise one or more apparatuses connected in parallel, whereby these are preferably characterised by an identical architecture.

Under the term “apparatuses” are understood both fixed-bed synthesis reactors as well as fixed-bed synthesis reactors with its own respective product separations.

In preferred embodiments of the present invention, one or more further reaction stages are connected serially downstream of the first and/or second reaction stage, comprising a fixed-bed synthesis reactor and a product separation.

In some variants, it is therefore possible to serially connect downstream of the first and second fixed-bed synthesis reactors further first and second synthesis reactors, preferably further fixed-bed synthesis reactors. Furthermore, it is conceivable that one or more synthesis reactors, preferably further fixed-bed synthesis reactors, are connected in parallel to the first and second fixed-bed synthesis reactors in order to increase the overall capacity of the plant. These reactors connected in parallel may each be provided with their own product separations, or the product stream may be combined prior to product separation and then passed through a common product separation.

In the context of the present invention, synthesis gas is added exclusively to the first fixed-bed synthesis reactor. The mixture comprising short-chain and shorter-chain hydrocarbons exiting the first fixed-bed synthesis reactor and optionally processed via a product separation is not considered as synthesis gas in the context of the present invention, even if it contains hydrogen and carbon monoxide.

Common catalysts used in FTS include the transition metals cobalt, nickel, iron and/or ruthenium. Catalysts containing various mixtures of the aforementioned metals or promoters, for example from the lanthanide group, are also known and used for the reaction. As supports usually high temperature stable materials, which Al₂O₃, ZrO₂, SiO₂, TiO₂, various ceramics or mixtures of these, are used.

In the context of the present invention, such common supported or unsupported catalysts are used, with the proviso that they contain cobalt as catalytically active component.

The optimum amount of catalytically active metal, i.e. cobalt, depends on the support material used. Typically, the content of cobalt in the catalysts used in the context of the present invention is between 1 and 100 parts by weight per 100 parts by weight of support material, preferably between 10 and 50 parts by weight per 100 parts by weight of support material.

Concomitantly, the catalysts used in the context of the present invention may further comprise one or more metallic promoters or co-catalysts. These may be present as metal or as metal oxides. Suitable promoters include oxides of metals of Groups IIA, IIIB, IVB, VB, VIB and VIIB of the Periodic Table of the Elements and oxides of lanthanides and/or actinides. For example, based on titanium, zirconium, manganese and/or vanadium. Alternatively or in addition to the metal oxide promoters, the catalysts may comprise metallic promoters selected from Groups VIIB and/or VIII of the Periodic Table of the Elements. For example, rhenium, platinum and/or palladium. Typically, the promoter content, if any, in the catalysts used in the present invention is between 0.1 and 60 parts by weight per 100 parts by weight of support material, this content may vary widely within the mentioned limits depending on the exact promoter material used.

A catalyst based on cobalt as the catalytically active metal and comprising manganese and/or vanadium as promoters is well suited. An example of this is a catalyst in which the atomic ratio of cobalt to promoter is at least 12:1.

It is essential to the present invention that the same cobalt-based Fischer Tropsch catalyst is used in all reactors.

The size of the catalyst particles used in the present invention also depends on the exact reactor. For example, in microreactors often catalysts with smaller particle sizes are used.

Accordingly, in some variants of the present invention, catalysts having an average diameter of 0.5 mm to 15 mm are used.

The catalysts can also be extrudates, in which case they have, for example, a length of 2 mm to 10 mm, in particular 5 mm to 6 mm, and a cross-sectional area of 1 to 6 mm2, preferably 2 to 3 mm2.

Examples of commercially available catalysts that can be used in the context of the present invention are described, for example, in WO 2011/06184 A1.

The weight ratio of the catalyst amounts, in a process with two fixed-bed synthesis reactors, a weight ratio between 1.1:1 and 4.3:1, preferably 1.2:1 and 4.3:1, catalyst amount in the first fixed-bed synthesis reactor to catalyst amount in the second fixed-bed synthesis reactor is set in the context of the present invention. In particularly preferred variants, the weight ratio is set at 1.25:1 to 2.5:1.

A particularly preferred weight ratio in the context of the present invention is 2:1.

If one sets the weight ratio of the catalysts to a specific ratio, the possibility arises to control the desired CO conversion rate by an adjustment of the reactor temperature.

The origin of the synthesis gas is in principle not limited. For example, the synthesis gas can be obtained from gasification of biomass, from synthesis gas generation from fossil starting materials (natural gas, crude oil, coal), or from electricity-based processes (conversion of electrolytically generated H₂ as well as CO₂).

In preferred embodiments of the present invention, the product separation is carried out in multiple stages. More suitably, a multi-stage product separation comprises at least one hot separator and one cold separator.

For example, in some variants of the present invention, the hot separator is operated at a temperature of 160 to 200° C., for example about 180° C., and cold separator is operated at a temperature of 0 to 20° C., for example 10° C. These ranges apply in particular both to the first separation stage as well as to the subsequent ones, in order to be able to obtain fractions corresponding to each other.

The advantage of such a multi-stage product separation is that the individual product groups of the FTS have different boiling temperatures, which can be exploited for the separation. By precisely setting the temperature levels within the individual stages of product separation, a targeted separation of the desired products is possible. With an increasing number of stages, an improved separation can also be observed. As an example of a product separation with numerous stages, a rectification column can be mentioned.

In preferred embodiments of the present invention, water is additionally separated during the product separation.

This has the advantage that the water which is harmful to the catalyst can be separated between the stages and thus the catalytic reaction in the second synthesis reactor is not impaired by the water. The separated reaction water can be reused in the process.

In the method of the present invention, the molar ratio of H₂ to CO in the synthesis gas is adjusted to a ratio of 1.7:1 to 2.3:1, preferably 1.8:1 to 2.3:1, particularly preferably 1.9:1 to 2.3:1. In variants of the present invention, it is adjusted to a molar ratio selected from the group consisting of the ratios 1.8:1, 1.9:1, 2.0:1, 2.1:1, 2.2:1 and 2.3:1. In this regard, it should be appreciated that while certain preferred ratios are mentioned herein, the present invention is not limited to these. Of course, the present invention also encompasses ratios lying between these values.

By sub-stoichiometric operation, longer hydrocarbon chains are formed, the undesirable methane selectivity falls and thus less H₂ per CO molecule is required overall. More CO can thus be converted to target products per H₂ used. The selectivity with regard to the formation of long-chain hydrocarbons increases.

The first fixed-bed synthesis reactor is preferably operated in such a way that the selectivity for the end products (preferably long-chain hydrocarbons in certain quantities also (terminal) alkenes) is particularly high. The double bond present enables further growth of the hydrocarbon chain in the subsequent second fixed-bed reactor stage through readsorption of the hydrocarbons on the catalyst. Unsaturated long chain hydrocarbons separated in the product separation may require subsequent treatment with hydrogen to hydrogenate the double bond(s).

The preferred goal of the operation of the second fixed-bed synthesis reactor is the reaction of remaining synthesis gas and the conversion of the short-chain and shorter-chain hydrocarbons from the first fixed-bed synthesis reactor to proportionally as many long-chain hydrocarbons as possible. In addition to this target product, shorter-chain hydrocarbons (chain length: C₅-C₂₄) and a gas fraction of light, short-chain hydrocarbons (C₁-C₄) and residual gases (CO, CO₂, H₂) are produced in the second product separation. Most of the oxygen-containing hydrocarbons (by-products: alcohols, organic acids, . . . ) are dissolved in the aqueous phase.

The reaction conditions in the first and second fixed-bed synthesis reactors, as well as all further fixed-bed synthesis reactors, are adjusted within the scope of the present invention for conversion control by controlling the reactor temperature to an equal value between 180° C. and 250° C. in all synthesis reactors depending on the desired, between 40 and 90 mol %, total CO conversion.

In preferred embodiments of the present invention, the reactor temperatures in all fixed-bed synthesis reactors are controlled to an equal value between 200 and 240° C., particularly preferably 200 to 230° C., in particular preferably 200 to 220° C., even more preferably 200 to 210° C., wherein the values are to be considered with a tolerance of plus/minus 3° C., respectively.

In variants, the temperature can be set to a value selected from the group consisting of 200° C., 205° C., 210° C., 215° C., 220° C., 225° C., 230° C., 235° C. and 240° C. In this regard, it should be appreciated that while certain preferred temperature values are mentioned herein, the present invention is not limited to these. Of course, the present invention also encompasses temperatures lying between these values; the values mentioned are merely simple control steps. Stepless control is equally possible.

The inert gas content of the synthesis gas fed in the context of the present invention is between 0 vol. % and 50 vol. %. It is preferred if the inert gas content is between 0 and 40 vol. %. Specific values for the inert gas content in the synthesis gas are selected in variants of the present invention from the group consisting of 0 vol. %, 5 vol. %, 10 vol. %, 15 vol. %, 20 vol. %, 25 vol. %, 30 vol. %, 35 vol. % and 40 vol. %. In this regard, it should be appreciated that while certain preferred percentages are mentioned herein, the present invention is not limited to these. Of course, the present invention also encompasses percentages lying between these values.

The weight volume flow rate (WHSV(CO)) for Fischer-Tropsch syntheses in the context of the present invention can, for example, be set to values between 0.1 and kgCO/(kgKat*h). It is essential that it is set to an input value and this is then not changed during the ongoing process, but left constant during the process. There is also no readjustment in this respect between the individual stages. Minor fluctuations caused by the equipment in the weight volume flow at the input are harmless.

In preferred embodiments of the present invention, the first fixed-bed synthesis reactor is operated at a pressure of 15 to 30 bar, preferably 19 to 25 bar, in particular 22 bar and, independently thereof, and the second fixed-bed synthesis reactor at a pressure of 15 to 30 bar, preferably 17 to 23 bar, in particular 20 bar.

It is possible in the context of the present invention that all reactor stages are operated at the same pressure and are set to the same pressure. It is also possible that the pressure in the first reactor is lower than in the following reactor(s).

It is preferred, in the context of the present invention, if the first reactor operates at the highest pressure and the subsequent reactors each have a slightly lower pressure than the immediately preceding reactor.

In preferred embodiments, the pressure in the entire apparatus is adjusted by a single pressure control device, in particular located downstream of the last reactor. This is an example for one possibility of adjusting the preferred pressure distribution of the present invention, namely that the first reactor operates at the highest pressure and the subsequent reactors each have a slightly lower pressure than the immediately preceding reactor.

In particularly preferred embodiments of the present invention, the molar H₂:CO ratio in the synthesis gas, the inert gas proportion in the synthesis gas, the quantitative ratio of the catalysts to each other, the pressure in the first fixed-bed synthesis reactor and the pressure in the second fixed-bed synthesis reactor, as well as the weight volume flow rate are kept constant.

For these embodiments, the control of the conversion is in particular possible very precisely. It is not mandatory to keep all these parameters constant. However, in this way the control is best. In particular, in this way the synthesis process is very well controllable and easy to monitor. This represents a great advantage in terms of equipment and organisation, because an effective and reliable process control is possible via a few controllers and with few personnel. Automation is also much easier to realise in this case.

It is highly preferred in the context of the present invention to set the molar H₂:CO ratio to a value within the ranges mentioned and to keep it constant during the process, in particular to a ratio between 1.9 and 2.4, preferably 2.0 and 2.4, more preferably 2.0 to 2.3, in particular 2.1.

In preferred embodiments, the values are to be regarded in each case with a tolerance of plus/minus 0.3, particularly preferably with a tolerance of 0.1, in particular without tolerance, i.e. only with fluctuations due to measurement technology.

In other alternatives of the present invention, it is highly preferred, in the context of the present invention, to set the molar H₂:CO ratio to a value within the ranges mentioned and to keep it constant during the process, in particular to a ratio between 1.9 and 2.3, preferably 2.0 and 2.3, more preferably 2.1 to 2.3, even more preferably 2.2 to 2.3 and most preferably 2.3. In some preferred embodiments, the values are in each case to be regarded as having a tolerance of plus/minus 0.1, preferably 0.05, in particular without tolerance, i.e. only with fluctuations due to measurement technology.

In the context of the present invention, it is also essential that it is controlled such that the hydrogen conversion, considered over all stages, it is at most 99 mol %, preferably at most 98 mol %, particularly preferably at most 97 mol %, especially preferably at most 96 mol % and most preferably at most 95 mol %.

By controlling for a non-complete conversion of the hydrogen, on the one hand it is prevented that carbon is formed and precipitates on the reactor walls or the catalyst and on the other hand a re-oxidation of the catalyst is prevented, which would lead to a deactivation of the catalyst. It should be taken into account that carbon is not necessarily formed immediately even above 95 mol % conversion, but according to the invention this value is controlled in order to be able to continue to ensure a stable process flow.

Accordingly, in variants of the present invention, a hydrogen conversion of at most 98 mol %, or at most 97 mol %, or at most 96 mol %, or at most 95 mol %, considered over all stages, is controlled.

In preferred embodiments of the present invention, a product stream leaving the second fixed-bed synthesis reactor comprising long chain hydrocarbons is fed to a second product separation to separate a fraction of long chain hydrocarbons from the product stream.

In this second product separation, water is preferably also separated. Thus, the product stream leaving the second product separation, comprising short-chain hydrocarbons can be fed to a further fixed-bed synthesis reactor.

The process according to the invention is advantageous with regard to the following points, among others.

Due to the possible use of a wide variety of educt gas streams of fossil as well as renewable origin, which are only limited by the mentioned molar H₂:CO ratios and the inert gas content, a wide range of applications is available. In particular, the yield of the target product can be maximised in the power-to-liquid process, in which CO 2 is converted into the target product together with renewable, electrolytically produced hydrogen. This is particularly important because the energetically expensive process routes for providing the reactants, especially H₂ via electricity-based processes such as electrolysis, or CO 2 in the case of capture from e.g. the air, require a most efficient and targeted conversion possible into target product so that these processes can be carried out in an economically attractive manner. The efficiency of a power-to-liquid plant is measured in terms of the amount of target product per electricity expended.

Furthermore, conversion control can be implemented relatively easily through the measures mentioned above. In most multi-stage plants according to the state of the art, a simple intermediate separation of the products of the first reactor is already provided for. The continuation of the C₅-C₂₄ fraction into the next stage is thus possible with only minor modifications to existing systems. The increase in the yield of long-chain hydrocarbons, in particular of the very valuable C₂₅ hydrocarbons, can thus be increased by adapted reaction and separation conditions with relatively little effort.

Particularly preferred variants of the present invention relate to a conversion of 50 to 60%, a molar H₂:CO ratio of between 1.9:1 and 2.3:1, an inert gas content of 0 to 40 vol. % and a weight ratio of the catalysts of 1.25:1 to 2.52:1, as well as a pressure in the first reactor of 18 to 26 bar and in the second reactor 16 to 24 bar.

The present invention also relates to a plant for carrying out the method described above, comprising
i) a first fixed-bed synthesis reactor comprising a cobalt-based Fischer-Tropsch catalyst,
ii) a single- or multi-stage product separation serially connected downstream of the first fixed-bed synthesis reactor, which is adapted to at least
a) separate a fraction of hydrocarbons from a product stream leaving the first fixed-bed synthesis reactor,
b) optionally separate water in addition to the hydrocarbons,
iii) a second fixed-bed synthesis reactor serially connected downstream of the product separation comprising the same catalyst as in fixed-bed synthesis reactor, wherein the plant is configured such that synthesis gas addition is exclusively to the first fixed-bed synthesis reactor, characterized in that the weight ratio of catalyst from the first fixed-bed synthesis reactor to the second fixed-bed synthesis reactor is between 1.2:1 and 4.3:1, preferably between 1.25:1 and 2.52:1, more preferably 2:1.

In preferred embodiments, the plant of the present invention comprises a further product separation A) serially connected downstream of the second fixed-bed synthesis reactor, which is designed to separate a fraction of long-chain hydrocarbons from a product stream leaving the second fixed-bed synthesis reactor.

In preferred embodiments of the plant of the present invention, each fixed-bed synthesis reactor may comprise one or more apparatuses B) connected in parallel, wherein these are preferably characterised by an identical architecture.

In preferred embodiments, the plant of the present invention comprises one or more further reaction stages C) which are connected in series downstream of the first and/or second reaction stage comprising a fixed-bed synthesis reactor as well as a product separation.

The above-mentioned additional features A), B) and C) of the plant of the present invention may be implemented each for itself, in any combination or all together.

The preferred embodiments described above for the method according to the invention and the advantages associated therewith apply analogously to the plant according to the invention.

For details of the individual devices or units of the plant, reference is made to the above details in this respect. The above details for the method apply accordingly to the plant.

The first and/or the second fixed-bed synthesis reactor is preferably a micro structured fixed-bed synthesis reactor.

The first and the second fixed-bed synthesis reactor preferably have the same architecture.

The person skilled in the art can make the exact design of the reactor, such as size, wall thicknesses, materials, etc., to the reaction conditions advised for a particular reaction within the scope of his general skill in the art.

Where, in the description of the plant according to the invention, parts or the entire plant are designated as “consisting of”, this is to be understood as referring to the essential components mentioned. Self-evident or inherent parts such as pipes, valves, screws, housings, measuring devices, storage tanks for reactants/products etc. are not excluded by this. Preferably, however, other essential components, such as additional reactors or the like, which would change the process sequence, are excluded.

The individual parts of the system are in effective connection with each other in a customary and known manner.

Furthermore, a subject matter of the present invention is a method for controlling the CO conversion in multi-stage Fischer-Tropsch syntheses, in which synthesis gas is only added to the first synthesis reactor, to between 40 and 90 mol %, preferably 50 to 80%, in particular 50 to 60 mol %, by continuously and simultaneously adjusting the reactor temperatures for all Fischer-Tropsch synthesis reactors to an equal value between 180° C. and 250° C., wherein the weight volume flow at the inlet of the FTS is adjusted to a value and kept constant at this value during the process, wherein preferably the parameters mentioned below are set and kept constant during the synthesis process: molar H₂:CO ratio in the synthesis gas of 1.7:1 to 2.3:1, inert gas content in the synthesis gas between 0 and 40 vol. %, same cobalt-based Fischer-Tropsch catalyst in all reactors, weight ratio of the amount of catalyst of first fixed-bed synthesis reactor to second fixed-bed synthesis reactor between 1.2:1 and 4.3:1, pressure in the fixed-bed synthesis reactors 10 to 50 bar in each case, hydrogen conversion considered over all stages at most 99 mol %.

The above explanations regarding the individual features, process steps and preferred embodiments apply mutatis mutandis to this method.

In this method, the CO conversion is controlled to the desired value by adjusting the reaction temperature in all reactors to the same temperature. A prerequisite in the context of the present invention is that the other parameters mentioned are kept constant.

It is also particularly advantageous here that in this way the course of the reaction can be kept very constant and the resulting amount of valuable product can be planned precisely. In addition, with this method it is possible to change the product distribution in a targeted manner during the ongoing process if, for example, a certain fraction of the product mixture is under- or over-represented compared to the currently desired ratio.

Finally, it is still a subject matter of the present invention to provide a method for compensation of catalyst deactivation in multistage continuously operating Fischer-Tropsch syntheses in which synthesis gas addition is exclusively to the first synthesis reactor, by continuous and simultaneous adjustment of the reactor temperatures for all Fischer-Tropsch synthesis reactors to an equal value between 180° C. and 250° C., whereby the weight volume flow at the inlet of the FTS is adjusted to a value and kept constant at this value during the process, whereby preferably the parameters mentioned below are adjusted and kept constant during the synthesis process: molar H₂:CO ratio in the synthesis gas of 1.7:1 to 2.3:1, inert gas content in the synthesis gas between 0 and 40 vol. %, the same cobalt-based Fischer-Tropsch catalyst in all reactors, weight ratio of the amount of catalyst in the first fixed-bed synthesis reactor to the second fixed-bed synthesis reactor of between 1.2:1 and 4.3:1, pressure in the fixed-bed synthesis reactors of 10 to 50 bar in each case, hydrogen conversion considered over all stages at most 99 mol %, CO conversion in the stages of between and 90 mol %, preferably 50 to 80 mol %, in particular 50 to 60 mol %.

The explanations given above with respect to the individual features, process steps and preferred embodiments also apply mutatis mutandis to this method.

Particularly advantageous in the present invention is that in this way the course of the reaction can be kept very constant, and the resulting amount of valuable product can be precisely planned. In addition, with this method it is possible to selectively change the product distribution during the ongoing process if, e.g., a certain fraction of the product mixture is under- or over-represented compared to the currently desired ratio.

The control of CO conversion according to the present invention is an immense advantage in terms of equipment and process technology, as it is relatively easy to achieve cooling of all reactors to the same temperature. This can be achieved, for example, by selectively arranging all reactors in a heat exchanger complex.

Another surprising and advantageous effect of the present invention is that a very good reaction and process control is possible, although there is no addition of synthesis gas after the first reactor. Contrary to expectations, good controllability is achieved despite the lack of intermediate stage control or intermediate stage readjustment. Based on the prior art, it was not to be expected that by the measures according to the invention or the procedure according to the invention a precise control of the Fischer-Tropsch synthesis in a simple manner would be possible.

The control of the conversion or the possibility to keep the conversion specifically adjusted to a constant value is very effective with the method according to the invention and the plant according to the invention and much simpler than an adjustment of the catalyst amounts.

By the present invention a high hydrogen conversion is achieved, while at the same time a safe operating condition is ensured.

It is advantageous in the present invention, that the weight volume flow is kept constant at the inlet of the AGV. This is a great advantage in that by this and the control via the temperature the integration into further (industrial) processes is considerably facilitated. For it is not at all unusual that other processes, for example synthesis gas production, provide a constant weight volume flow. In the context of the present invention, this can then simply be fed directly on to the FTS.

Based on the known prior art, it was in particular unexpected that a good and simple control of multi-stage FTS with constant weight volume flow at the inlet via temperature is possible, whereby at the same time very good results can be achieved.

Although the present invention is described in this description essentially with reference to two fixed-bed synthesis reactors, the present invention is expressly also related to methods and plants comprising more than two fixed-bed synthesis reactors, wherein a fixed-bed synthesis reactor is then each time followed by a product separation apparatus or product separation step. In particular also encompassed by the present invention are multi-stage processes and plants with five fixed-bed synthesis reactors, four fixed-bed synthesis reactors, or three fixed-bed synthesis reactors.

The various embodiments of the present invention, e.g., but not limited to, those of the various dependent claims, may thereby be combined with each other in any manner, provided that such combinations do not contradict each other.

### DESCRIPTION OF THE FIGURES

The present invention is explained in more detail below with reference to the drawings. The drawings are not to be construed as limiting and are not to scale. The drawings are schematic and furthermore do not contain all the features that conventional devices have, but are reduced to the features that are essential for the present invention and its understanding, for example, screws, connections etc. are not shown or not shown in detail.

Identical reference signs indicate identical features in the figures, the description and the claims.

FIG. 1:

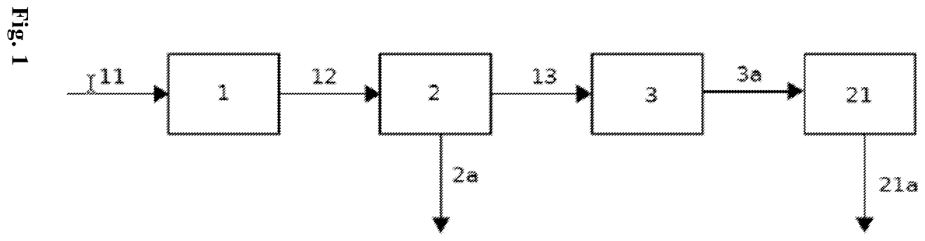

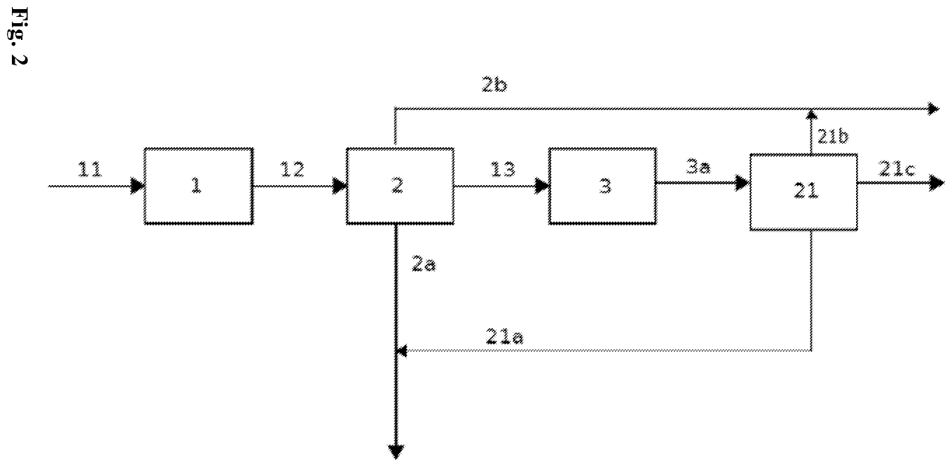

A synthesis gas stream comprising H₂ and CO 11 is fed at a constant weight volume flow into a first fixed-bed synthesis reactor 1, in which H₂ and CO are catalytically converted to hydrocarbons. A product stream 12 leaving the first fixed-bed synthesis reactor 1 is fed into a (first) separation device 2, in which a fraction of long-chain hydrocarbons is separated 2a. The remaining fractions, comprising essentially short and shorter chain hydrocarbons, CO, CO₂ and H₂, as well as possibly residues of H₂O 13, are fed into a second fixed-bed synthesis reactor 3, and catalytically converted to long chain hydrocarbons (>C₂₅) 3a.

FIG. 2:

A synthesis gas stream comprising H₂ and CO 11 is fed at a constant weight volume flow into a first fixed-bed synthesis reactor 1, in which H₂ and CO are catalytically converted to hydrocarbons. A product stream 12 leaving the first fixed-bed synthesis reactor 1 is fed into a (first) separation device 2, in which a fraction of long-chain hydrocarbons, as well as an aqueous fraction is separated 2a. The remaining fractions, comprising essentially short and shorter chain hydrocarbons, CO, CO₂ and H₂ 13 are fed into a second fixed-bed synthesis reactor 3, and catalytically converted to essentially long chain hydrocarbons. A product stream 3a of the second fixed-bed synthesis reactor 3 is fed to a second product separation 21, in which the fraction of long chain hydrocarbons 21a is separated from the fraction comprising short and shorter chain hydrocarbons (C₁-C₂₄), CO, CO₂ and H₂, 21c as well as an aqueous fraction 21b. The aqueous fraction 21b can be combined with the aqueous fraction from the first product separation 2b. The fraction of long-chain hydrocarbons 21a separated by means of the second product separation is combined with the fraction of long-chain hydrocarbons 2a from the first product separation.

### LIST OF REFERENCE SIGNS

1 first fixed-bed synthesis reactor
11 synthesis gas stream comprising H₂ and CO
12 product stream of the first fixed-bed synthesis reactor
2 (first) product separation
2a fraction of separated hydrocarbons
2b aqueous phase
21 second product separation
21a fraction of separated hydrocarbons
21b aqueous phase
21c fraction comprising short and/or shorter chain hydrocarbons, CO, CO₂ and H₂
13 fraction comprising short and/or shorter chain hydrocarbons, CO, CO₂ and H₂, as well as optionally H₂O
3 second fixed-bed synthesis reactor
3a product stream of the second fixed-bed synthesis reactor (enriched with long chain hydrocarbons (>C₂₅)).

### EXAMPLES

The invention will now be further explained with reference to the following non-limiting examples.

### Example 1

FTS was carried out with two reactors connected in sequence according to the invention, each using the same cobalt-based catalyst. In each case, temperature, target conversion, H₂:CO ratio, inert gas content were set differently and the individual results were tabulated, wherein the values in the table indicate the required catalyst mass ratio of catalyst mass in the first fixed-bed synthesis reactor to catalyst mass in the second fixed-bed synthesis reactor in order to achieve the respective conversion as a function of temperature.

During the respective tests, a pressure of 22 bar prevailed in the first reactor and 20 bar in the second reactor and the weight volume flow of the synthesis gas stream added remained constant.

The following table shows a design matrix in which the experimental data of the process discussed above are entered. The matrix has been divided into several pages for better readability.

The temperature was outlined in steps of 200° C., 210° C., 220° C., 230° C. and 240° C. against the molar CO conversion in steps of 50 mol %, 60 mol %, 70 mol %, 80 mol %.

In each sub-table, the molar H₂:CO ratio was outlined in steps of 1.8:1 1.9:1 2.0:1 2.1:1, 2.2:1, 2.3:1 against the inert gas fraction in steps of 0%, 5%, 10%, 15%, 20%, 25%, 30%, 35%, 40%.

Values marked with an asterisk (*) are values where the hydrogen conversion increased to above 99 mol %. If the values in the table are marked with “0*”, this means that complete hydrogen conversion already took place in the first stage.

It can be seen here that the optimal ratio of the catalyst amounts is between 2.52:1 and 1.25:1.

In addition, it is easy to see that the reaction can be easily controlled by adjusting the temperature.

| | | temperature | | | | | | | | | | | | | |
|---|---|---|---|---|---|---|---|---|---|---|---|---|---|---|---|
| | | 200° C. | | | | | | | 210° C. | | | | | | |
| | | | 1.8 | 1.9 | 2.0 | 2.1 | 2.2 | 2.3 | | 1.8 | 1.9 | 2.0 | 2.1 | 2.2 | 2.3 |
| conversion | 50% | 0 | 2.0 | 1.9 | 1.9 | 1.8 | 1.7 | 1.7 | 0 | 2.0 | 1.9 | 1.9 | 1.8 | 1.8 | 1.7 |
| CO | | 5 | 1.9 | 1.8 | 1.8 | 1.7 | 1.7 | 1.6 | 5 | 1.9 | 1.8 | 1.8 | 1.7 | 1.7 | 1.6 |
| | | 10 | 1.8 | 1.7 | 1.7 | 1.6 | 1.6 | 1.5 | 10 | 1.8 | 1.7 | 1.7 | 1.6 | 1.6 | 1.6 |
| | | 15 | 1.7 | 1.6 | 1.6 | 1.6 | 1.5 | 1.5 | 15 | 1.7 | 1.6 | 1.6 | 1.6 | 1.5 | 1.5 |
| | | 20 | 1.6 | 1.6 | 1.5 | 1.5 | 1.5 | 1.4 | 20 | 1.6 | 1.6 | 1.5 | 1.5 | 1.5 | 1.4 |
| | | 25 | 1.5 | 1.5 | 1.5 | 1.4 | 1.4 | 1.4 | 25 | 1.5 | 1.5 | 1.5 | 1.4 | 1.4 | 1.4 |
| | | 30 | 1.5 | 1.4 | 1.4 | 1.4 | 1.4 | 1.3 | 30 | 1.5 | 1.4 | 1.4 | 1.4 | 1.4 | 1.3 |
| | | 35 | 1.4 | 1.4 | 1.3 | 1.3 | 1.3 | 1.3 | 35 | 1.4 | 1.4 | 1.4 | 1.3 | 1.3 | 1.3 |
| | | 40 | 1.3 | 1.3 | 1.3 | 1.3 | 1.3 | 1.2 | 40 | 1.3 | 1.3 | 1.3 | 1.3 | 1.3 | 1.3 |
| | 60% | 0 | 2.7 | 2.5 | 2.4 | 2.2 | 2.1 | 2.0 | 0 | 2.7 | 2.5 | 2.4 | 2.2 | 2.1 | 2.0 |
| | | 5 | 2.4 | 2.3 | 2.2 | 2.1 | 2.0 | 1.9 | 5 | 2.4 | 2.3 | 2.2 | 2.1 | 2.0 | 1.9 |
| | | 10 | 2.2 | 2.1 | 2.0 | 1.9 | 1.8 | 1.8 | 10 | 2.2 | 2.1 | 2.0 | 1.9 | 1.9 | 1.8 |
| | | 15 | 2.0 | 1.9 | 1.9 | 1.8 | 1.7 | 1.7 | 15 | 2.0 | 1.9 | 1.9 | 1.8 | 1.8 | 1.7 |
| | | 20 | 1.9 | 1.8 | 1.7 | 1.7 | 1.6 | 1.6 | 20 | 1.9 | 1.8 | 1.8 | 1.7 | 1.7 | 1.6 |
| | | 25 | 1.7 | 1.7 | 1.6 | 1.6 | 1.6 | 1.5 | 25 | 1.7 | 1.7 | 1.7 | 1.6 | 1.6 | 1.5 |
| | | 30 | 1.6 | 1.6 | 1.5 | 1.5 | 1.5 | 1.5 | 30 | 1.6 | 1.6 | 1.6 | 1.5 | 1.5 | 1.5 |
| | | 35 | 1.5 | 1.5 | 1.5 | 1.4 | 1.4 | 1.4 | 35 | 1.5 | 1.5 | 1.5 | 1.5 | 1.4 | 1.4 |
| | | 40 | 1.5 | 1.4 | 1.4 | 1.4 | 1.4 | 1.3 | 40 | 1.5 | 1.4 | 1.4 | 1.4 | 1.4 | 1.3 |
| | 70% | 0 | 0.0* | 3.5 | 3.2 | 2.9 | 2.7 | 2.5 | 0 | 0.0* | 3.5 | 3.2 | 2.9 | 2.7 | 2.5 |
| | | 5 | 0.0* | 3.0 | 2.8 | 2.6 | 2.4 | 2.3 | 5 | 0.0* | 3.0* | 2.8 | 2.6 | 2.4 | 2.3 |
| | | 10 | 0.0* | 2.6 | 2.4 | 2.3 | 2.2 | 2.1 | 10 | 0.0* | 2.5* | 2.5 | 2.3 | 2.2 | 2.1 |
| | | 15 | 0.0* | 2.3 | 2.2 | 2.1 | 2.0 | 1.9 | 15 | 0.0* | 2.3* | 2.2 | 2.1 | 2.0 | 2.0 |
| | | 20 | 0.0* | 2.1 | 2.0 | 1.9 | 1.9 | 1.8 | 20 | 0.0* | 0.0* | 2.0 | 1.9 | 1.9 | 1.8 |
| | | 25 | 0.0* | 1.9* | 1.8 | 1.8 | 1.7 | 1.7 | 25 | 0.0* | 0.0* | 1.9 | 1.8 | 1.8 | 1.7 |
| | | 30 | 0.0* | 1.9* | 1.7 | 1.7 | 1.6 | 1.6 | 30 | 0.0* | 0.0* | 1.7 | 1.7 | 1.6 | 1.6 |
| | | 35 | 0.0* | 1.7* | 1.6 | 1.6 | 1.5 | 1.5 | 35 | 0.0* | 0.0* | 1.6 | 1.6 | 1.5 | 1.5 |
| | | 40 | 0.0* | 0.0* | 1.5 | 1.5 | 1.4 | 1.4 | 40 | 0.0* | 0.0* | 1.5 | 1.5 | 1.5 | 1.4 |
| | 80% | 0 | 0.0* | 0.0* | 5.0 | 4.2 | 3.7 | 3.3 | 0 | 0.0* | 0.0* | 0.0* | 4.3 | 3.7 | 3.4 |
| | | 5 | 0.0* | 0.0* | 3.8* | 3.4 | 3.1 | 2.8 | 5 | 0.0* | 0.0* | 0.0* | 3.4 | 3.1 | 2.9 |
| | | 10 | 0.0* | 0.0* | 3.2* | 2.9 | 2.7 | 2.5 | 10 | 0.0* | 0.0* | 0.0* | 2.9 | 2.7 | 2.5 |
| | | 15 | 0.0* | 0.0* | 2.5* | 2.5 | 2.3 | 2.2 | 15 | 0.0* | 0.0* | 0.0* | 2.5 | 2.4 | 2.3 |
| | | 20 | 0.0* | 0.0* | 2.6* | 2.2 | 2.1 | 2.0 | 20 | 0.0* | 0.0* | 0.0* | 2.3 | 2.1 | 2.1 |
| | | 25 | 0.0* | 0.0* | 0.0* | 2.0 | 1.9 | 1.9 | 25 | 0.0* | 0.0* | 0.0* | 2.0 | 2.0 | 1.9 |
| | | 30 | 0.0* | 0.0* | 0.0* | 1.8 | 1.8 | 1.7 | 30 | 0.0* | 0.0* | 0.0* | 1.9 | 1.8 | 1.7 |
| | | 35 | 0.0* | 0.0* | 0.0* | 1.7 | 1.6 | 1.6 | 35 | 0.0* | 0.0* | 0.0* | 1.7 | 1.7 | 1.6 |
| | | 40 | 0.0* | 0.0* | 0.0* | 1.6 | 1.5 | 1.5 | 40 | 0.0* | 0.0* | 0.0* | 1.6* | 1.6 | 1.5 |

| | | temperature | | | | | | | | | | | | | |
|---|---|---|---|---|---|---|---|---|---|---|---|---|---|---|---|
| | | 200° C. | | | | | | | 210° C. | | | | | | |
| | | | 1.8 | 1.9 | 2.0 | 2.1 | 2.2 | 2.3 | | 1.8 | 1.9 | 2.0 | 2.1 | 2.2 | 2.3 |
| conversion | 50% | 0 | 2.0 | 1.9 | 1.9 | 1.8 | 1.8 | 1.7 | 0 | 2.0 | 1.9 | 1.9 | 1.8 | 1.8 | 1.7 |
| CO | | 5 | 1.9 | 1.8 | 1.8 | 1.7 | 1.7 | 1.6 | 5 | 1.9 | 1.8 | 1.8 | 1.7 | 1.7 | 1.7 |
| | | 10 | 1.8 | 1.7 | 1.7 | 1.7 | 1.6 | 1.6 | 10 | 1.8 | 1.8 | 1.7 | 1.7 | 1.6 | 1.6 |
| | | 15 | 1.7 | 1.7 | 1.6 | 1.6 | 1.5 | 1.5 | 15 | 1.7 | 1.7 | 1.6 | 1.6 | 1.6 | 1.5 |
| | | 20 | 1.6 | 1.6 | 1.5 | 1.5 | 1.5 | 1.5 | 20 | 1.6 | 1.6 | 1.6 | 1.5 | 1.5 | 1.5 |
| | | 25 | 1.5 | 1.5 | 1.5 | 1.5 | 1.4 | 1.4 | 25 | 1.6 | 1.5 | 1.5 | 1.5 | 1.4 | 1.4 |
| | | 30 | 1.5 | 1.4 | 1.4 | 1.4 | 1.4 | 1.4 | 30 | 1.5 | 1.5 | 1.4 | 1.4 | 1.4 | 1.4 |
| | | 35 | 1.4 | 1.4 | 1.4 | 1.4 | 1.3 | 1.3 | 35 | 1.4 | 1.4 | 1.4 | 1.4 | 1.4 | 1.3 |
| | | 40 | 1.4 | 1.3 | 1.3 | 1.3 | 1.3 | 1.3 | 40 | 1.4 | 1.4 | 1.3 | 1.3 | 1.3 | 1.3 |
| | 60% | 0 | 2.7 | 2.5 | 2.4 | 2.3 | 2.2 | 2.1 | 0 | 2.6 | 2.5 | 2.4 | 2.3 | 2.2 | 2.1 |
| | | 5 | 2.4 | 2.3 | 2.2 | 2.1 | 2.0 | 1.9 | 5 | 2.4 | 2.3 | 2.2 | 2.1 | 2.0 | 2.0 |
| | | 10 | 2.2 | 2.1 | 2.0 | 1.9 | 1.9 | 1.8 | 10 | 2.2 | 2.1 | 2.0 | 2.0 | 1.9 | 1.8 |
| | | 15 | 2.0 | 2.0 | 1.9 | 1.8 | 1.8 | 1.7 | 15 | 2.0 | 2.0 | 1.9 | 1.8 | 1.8 | 1.7 |
| | | 20 | 1.9 | 1.8 | 1.8 | 1.7 | 1.7 | 1.6 | 20 | 1.9* | 1.8 | 1.8 | 1.7 | 1.7 | 1.7 |
| | | 25 | 1.8 | 1.7 | 1.7 | 1.6 | 1.6 | 1.6 | 25 | 1.7* | 1.7 | 1.7 | 1.7 | 1.6 | 1.6 |
| | | 30 | 1.6 | 1.6 | 1.6 | 1.5 | 1.5 | 1.5 | 30 | 1.7* | 1.6 | 1.6 | 1.6 | 1.5 | 1.5 |
| | | 35 | 1.6 | 1.5 | 1.5 | 1.5 | 1.4 | 1.4 | 35 | 0.0* | 1.6 | 1.5 | 1.5 | 1.5 | 1.4 |
| | | 40 | 1.5* | 1.5 | 1.4 | 1.4 | 1.4 | 1.4 | 40 | 0.0* | 1.5 | 1.5 | 1.4 | 1.4 | 1.4 |
| | 70% | 0 | 0.0* | 0.6* | 3.2 | 3.0 | 2.8 | 2.6 | 0 | 0.0* | 0.0* | 3.2 | 3.0 | 2.8 | 2.6 |
| | | 5 | 0.0* | 0.5* | 2.8 | 2.6 | 2.5 | 2.3 | 5 | 0.0* | 0.0* | 2.8 | 2.6 | 2.5 | 2.4 |
| | | 10 | 0.0* | 1.1* | 2.5 | 2.4 | 2.2 | 2.1 | 10 | 0.0* | 0.0* | 2.5 | 2.4 | 2.3 | 2.2 |
| | | 15 | 0.0* | 0.0* | 2.3 | 2.1 | 2.1 | 2.0 | 15 | 0.0* | 0.0* | 2.3 | 2.2 | 2.1 | 2.0 |
| | | 20 | 0.0* | 0.0* | 2.1 | 2.0 | 1.9 | 1.8 | 20 | 0.0* | 0.0* | 2.1 | 2.0 | 1.9 | 1.9 |
| | | 25 | 0.0* | 0.0* | 1.9 | 1.8 | 1.8 | 1.7 | 25 | 0.0* | 0.0* | 1.9* | 1.9 | 1.8 | 1.8 |
| | | 30 | 0.0* | 0.0* | 1.8 | 1.7 | 1.7 | 1.6 | 30 | 0.0* | 0.0* | 1.8* | 1.7 | 1.7 | 1.7 |
| | | 35 | 0.0* | 0.0* | 1.6 | 1.6 | 1.6 | 1.5 | 35 | 0.0* | 0.0* | 1.7* | 1.6 | 1.6 | 1.6 |
| | | 40 | 0.0* | 0.0* | 1.5 | 1.5 | 1.5 | 1.5 | 40 | 0.0* | 0.0* | 0.0* | 1.5 | 1.5 | 1.5 |
| | 80% | 0 | 0.0* | 0.0* | 0.7* | 4.3 | 3.8 | 3.4 | 0 | 0.0* | 0.0* | 0.0* | 4.3 | 3.9 | 3.5 |
| | | 5 | 0.0* | 0.0* | 0.0* | 3.5 | 3.2 | 2.9 | 5 | 0.0* | 0.0* | 0.0* | 3.6* | 3.2 | 3.0 |
| | | 10 | 0.0* | 0.0* | 0.0* | 3.0 | 2.8 | 2.6 | 10 | 0.0* | 0.0* | 0.0* | 3.2* | 2.8 | 2.6 |
| | | 15 | 0.0* | 0.0* | 0.0* | 2.6* | 2.4 | 2.3 | 15 | 0.0* | 0.0* | 0.0* | 2.7* | 2.5 | 2.4 |
| | | 20 | 0.0* | 0.0* | 0.0* | 2.3* | 2.2 | 2.1 | 20 | 0.0* | 0.0* | 0.0* | 0.0* | 2.2 | 2.1 |
| | | 25 | 0.0* | 0.0* | 0.0* | 2.2* | 2.0 | 1.9 | 25 | 0.0* | 0.0* | 0.0* | 0.3* | 2.0 | 2.0 |
| | | 30 | 0.0* | 0.0* | 0.0* | 1.6* | 1.8 | 1.8 | 30 | 0.0* | 0.0* | 0.0* | 2.6* | 1.9 | 1.8 |
| | | 35 | 0.0* | 0.0* | 0.0* | 1.6* | 1.7 | 1.7 | 35 | 0.0* | 0.0* | 0.0* | 0.0* | 1.8* | 1.7 |
| | | 40 | 0.0* | 0.0* | 0.0* | 0.0* | 1.6 | 1.6 | 40 | 0.0* | 0.0* | 0.0* | 0.0* | 1.7* | 1.6 |

| | | temperature | | | | | | |
|---|---|---|---|---|---|---|---|---|
| | | 240° C. | | | | | | |
| | | | 1.8 | 1.9 | 2.0 | 2.1 | 2.2 | 2.3 |
| conversion | 50% | 0 | 2.0 | 1.9 | 1.9 | 1.8 | 1.8 | 1.7 |
| CO | | 5 | 1.9 | 1.9 | 1.8 | 1.8 | 1.7 | 1.7 |
| | | 10 | 1.8 | 1.8 | 1.7 | 1.7 | 1.6 | 1.6 |
| | | 15 | 1.7 | 1.7 | 1.6 | 1.6 | 1.6 | 1.6 |
| | | 20 | 1.6 | 1.6 | 1.6 | 1.6 | 1.5 | 1.5 |
| | | 25 | 1.6 | 1.5 | 1.5 | 1.5 | 1.5 | 1.4 |
| | | 30 | 1.5 | 1.5 | 1.5 | 1.4 | 1.4 | 1.4 |
| | | 35 | 1.4 | 1.4 | 1.4 | 1.4 | 1.4 | 1.4 |
| | | 40 | 1.4 | 1.4 | 1.4 | 1.3 | 1.3 | 1.3 |
| | 60% | 0 | 2.5 | 2.5 | 2.4 | 2.3 | 2.2 | 2.1 |
| | | 5 | 2.3 | 2.3 | 2.2 | 2.1 | 2.0 | 2.0 |
| | | 10 | 1.8* | 2.1 | 2.1 | 2.0 | 1.9 | 1.9 |
| | | 15 | 0.6* | 2.0 | 1.9 | 1.9 | 1.8 | 1.8 |
| | | 20 | 1.3* | 1.9 | 1.8 | 1.8 | 1.7 | 1.7 |
| | | 25 | 0.3* | 1.8 | 1.7 | 1.7 | 1.6 | 1.6 |
| | | 30 | 0.2* | 1.7 | 1.6 | 1.6 | 1.6 | 1.5 |
| | | 35 | 0.1* | 1.6 | 1.5 | 1.5 | 1.5 | 1.5 |
| | | 40 | 0.0* | 1.5 | 1.5 | 1.5 | 1.4 | 1.4 |
| | 70% | 0 | 0.0* | 0.1 | 3.2 | 3.0 | 2.8 | 2.7 |
| | | 5 | 0.0* | 0.0* | 2.8 | 2.7 | 2.5 | 2.4 |
| | | 10 | 0.0* | 0.0* | 2.5* | 2.4 | 2.3 | 2.2 |
| | | 15 | 0.0* | 0.0* | 2.3* | 2.2 | 2.1 | 2.1 |
| | | 20 | 0.0* | 0.0* | 0.0* | 2.0 | 2.0 | 1.9 |
| | | 25 | 0.0* | 0.0* | 0.0* | 1.9 | 1.9 | 1.8 |
| | | 30 | 0.0* | 0.0* | 0.0* | 1.8 | 1.7 | 1.7 |
| | | 35 | 0.0* | 0.0* | 0.0* | 1.7 | 1.6 | 1.6 |
| | | 40 | 0.0* | 0.0* | 0.0* | 1.6* | 1.5 | 1.5 |
| | 80% | 0 | 0.0* | 0.0* | 0.0* | 0.0* | 3.9 | 3.5 |
| | | 5 | 0.0* | 0.0* | 0.0* | 0.0* | 3.3 | 3.1 |
| | | 10 | 0.0* | 0.0* | 0.0* | 0.0* | 2.9 | 2.7 |
| | | 15 | 0.0* | 0.0* | 0.0* | 0.0* | 2.5 | 2.4 |
| | | 20 | 0.0* | 0.0* | 0.0* | 0.0* | 2.4* | 2.2 |
| | | 25 | 0.0* | 0.0* | 0.0* | 0.0* | 2.3* | 2.0 |
| | | 30 | 0.0* | 0.0* | 0.0* | 0.0* | 2.1* | 1.9 |
| | | 35 | 0.0* | 0.0* | 0.0* | 0.0* | 0.1* | 1.7 |
| | | 40 | 0.0* | 0.0* | 0.0* | 0.0* | 0.0* | 1.6* |

1.-15. (canceled)

16. A method for operating a Fischer-Tropsch synthesis, wherein the method comprises:
I) feeding a synthesis gas containing H₂ and CO into a first fixed-bed synthesis reactor which comprises a first catalyst bed to form hydrocarbons by catalytic reaction,
II) feeding a product stream leaving the first fixed-bed synthesis reactor and comprising hydrocarbons to a product separation to separate a fraction of hydrocarbons from the product stream,
III) feeding a remaining fraction of the product stream, which comprises short chain and shorter chain hydrocarbons to a second fixed-bed synthesis reactor comprising a second catalyst bed to form long chain hydrocarbons by catalytic reaction,
wherein synthesis gas is exclusively fed to the first fixed-bed synthesis reactor and furthermore,
a weight volume flow of the synthesis gas introduced into the first fixed-bed synthesis reactor is adjusted to a value and kept constant at this value during the method,
a molar H₂:CO ratio in the synthesis gas is adjusted to a value of from 1.7:1 to 2.3:1,
an inert gas content in the synthesis gas is from 0% to 40 vol. %,
the same cobalt-based Fischer-Tropsch catalyst is used in both fixed-bed reactors,
a weight ratio of catalyst in the first fixed-bed synthesis reactor to catalyst in the second fixed-bed synthesis reactor is set to be from 1.1:1 to 4.3:1,
the first fixed-bed synthesis reactor is operated at a pressure of from 10 bar to 50 bar, and the second fixed-bed synthesis reactor is operated at a pressure from 10 bar to 50 bar,
and wherein a reactor temperature is controlled to an equal value of from 180° C. to 250° C. in both synthesis reactors depending on a desired total CO conversion, which is between 40 and 90 mol %, and the control is such that a hydrogen conversion, considered over all stages, is at most 99 mol %.

17. The method of claim 16, wherein the product separation is a multi-stage product separation.

18. The method of claim 16, wherein water is additionally separated in the product separation.

19. The method of claim 16, wherein the molar ratio of H₂ to CO in the synthesis gas is adjusted to a value of from 1.8:1 to 2.3:1.

20. The method of claim 16, wherein the molar ratio of H₂ to CO in the synthesis gas is adjusted to a value of from 2.0:1 to 2.3:1.

21. The method of claim 16, wherein the molar ratio of H₂ to CO in the synthesis gas is adjusted to a value of from 2.2:1 to 2.3:1.

22. The method of claim 16, wherein an inert gas content in the synthesis gas is selected from 0 vol. %, 5 vol. %, 10 vol. %, 15 vol. %, 20 vol. %, 25 vol. %, 30 vol. %, 35 vol. % and 40 vol. %.

23. The method of claim 16, wherein reactor temperatures in the first fixed-bed synthesis reactor and the second fixed-bed synthesis reactor are controlled to an equal value of from 200° C. to 240° C.

24. The method of claim 16, wherein reactor temperatures in the first fixed-bed synthesis reactor and the second fixed-bed synthesis reactor are controlled to an equal value of from 200° C. to 220° C.

25. The method of claim 16, wherein the first fixed-bed synthesis reactor is operated at a pressure of from 15 bar to 30 bar, and the second fixed-bed synthesis reactor is operated at a pressure of from 15 bar to 30 bar.

26. The method of claim 16, wherein
the molar H₂:CO ratio in the synthesis gas,
the inert gas content in the synthesis gas,
the weight ratio of the catalysts to each other,
the pressure in the first fixed-bed synthesis reactor and the pressure in the second fixed-bed synthesis reactor
are all kept constant.

27. The method of claim 16, wherein a product stream comprising hydrocarbons and leaving the second fixed-bed synthesis reactor is fed to a further product separation.

28. The method of claim 16, wherein one or both of the first and second fixed-bed synthesis reactors comprise two or more apparatuses connected in parallel.

29. The method of claim 16, wherein one or more further reaction stages comprising a fixed-bed synthesis reactor and a product separation are arranged serially downstream of the first and/or second reaction stage.

30. An installation, wherein the installation is suitable for carrying out the method of claim 16 and comprises:
i) a first fixed-bed synthesis reactor comprising a cobalt-based Fischer-Tropsch catalyst,
ii) a single- or multi-stage product separation arranged serially downstream of the first fixed-bed synthesis reactor and adapted to at least
a) separate a fraction of hydrocarbons from a product stream leaving the first fixed-bed synthesis reactor,
b) optionally, separate water in addition to the hydrocarbons,
iii) a second fixed-bed synthesis reactor arranged serially downstream of the product separation and comprising the same catalyst as in the first fixed-bed synthesis reactor,
and wherein the installation is configured such that synthesis gas addition is exclusively to the first fixed-bed synthesis reactor, a weight ratio of catalyst of first fixed-bed synthesis reactor to catalyst of second fixed-bed synthesis reactor being from 1.1:1 to 4.3:1.

31. The installation of claim 30, wherein the installation additionally comprises one or more of the following features:
A) a further product separation arranged serially downstream of the second fixed-bed synthesis reactor, which is designed to separate a fraction of long-chain hydrocarbons from a product stream leaving the second fixed-bed synthesis reactor,
B) each fixed-bed synthesis reactor comprises two or more apparatuses connected in parallel,
C) one or more further reaction stages are arranged serially downstream of a first and/or second reaction stage and comprise a fixed-bed synthesis reactor and product separation.

32. A method for controlling the CO conversion in a multistage Fischer Tropsch syntheses in which synthesis gas is added only to a first synthesis reactor, to a value of from 40% to 90 mol %, wherein the method comprises continuous and simultaneous adjustment of reactor temperatures for all Fischer-Tropsch synthesis reactors to an equal value of from 180° C. and 250° C., a weight volume flow of the synthesis gas introduced into the first fixed-bed synthesis reactor being adjusted to a value and kept constant at this value during the process.

33. The method of claim 32, wherein the following parameters are set and kept constant during the synthesis process:
molar H₂:CO ratio in the synthesis gas of from 1.7:1 to 2.3:1,
inert gas content in the synthesis gas from 0% and 40 vol. %,
same cobalt-based Fischer-Tropsch catalyst in all reactors,
weight ratio of amount of catalyst of first fixed-bed synthesis reactor to amount of catalyst of second fixed-bed synthesis reactor of from 1.2:1 to 4.3:1,
pressure in the fixed-bed synthesis reactors 10 bar to 50 bar each,
hydrogen conversion over all stages at most 99 mol %.

34. A method for the compensation of catalyst deactivation in a multistage continuously operating Fischer-Tropsch synthesis in which synthesis gas is added only to a first synthesis reactor, wherein the method comprises continuous and simultaneous adjustment of reactor temperatures for all Fischer-Tropsch synthesis reactors to an equal value of from 180° C. and 250° C., and setting a weight volume flow of the synthesis gas introduced into the first fixed-bed synthesis reactor to a value and keeping it constant at this value during the process.

35. The method of claim 34, wherein the following parameters are set and kept constant during the synthesis process:
molar H₂:CO ratio in the synthesis gas of from 1.7:1 to 2.3:1,
inert gas content in the synthesis gas of from 0% to 40 vol. %,
same cobalt-based Fischer-Tropsch catalyst in all reactors,
weight ratio of amount of catalyst of first fixed-bed synthesis reactor to amount of catalyst of second fixed-bed synthesis reactor of from 1.2:1 to 4.3:1,
pressure in the fixed-bed synthesis reactors 10 bar to 50 bar each,
hydrogen conversion over all stages at most 99 mol %,
CO conversion in each stage of from 40 and 90 mol %.
